(19) United States
(12) Patent Application Publication
(10) Pub. No.: US 2026/0079217 A1
(43) Pub. Date: Mar. 19, 2026

(54) CELL UNIT

(71) Applicant: HAMAMATSU PHOTONICS K.K., Hamamatsu-shi (JP)

(72) Inventor: Takeshi ENDO, Shizuoka (JP)

(73) Assignee: HAMAMATSU PHOTONICS K.K., Hamamatsu-shi (JP)

(21) Appl. No.: 19/318,736

(22) Filed: Sep. 4, 2025

(30) Foreign Application Priority Data
Sep. 6, 2024 (JP) 2024-154040

(51) Int. Cl. G01R 33/032; A61B 5/242

(52) U.S. Cl. CPC G01R 33/032; A61B 5/242; A61B 2562/16

(57) ABSTRACT

A cell unit includes a cell which includes a body portion through which laser light passes, and in which an alkali metal is sealed; a heat conductive member that is at least partially disposed on the body portion; and a heater thermally connected to the heat conductive member. The heat conductive member covers an outer surface of a wall portion of the body portion through which the laser light passes, and is transmissive to the laser light.

### TECHNICAL FIELD

The present disclosure relates to a cell unit used in, for example, an optically excited magnetic sensor.

### BACKGROUND

As an example of a cell unit used in an optically excited magnetic sensor, a cell unit including a cell which includes a body portion through which light passes, and in which an alkali metal is sealed, and a heater that generates heat for heating the body portion of the cell is known (for example, refer to Japanese Patent No. 5736795).

In the configuration described in Japanese Patent No. 5736795, the cell is covered with a covering layer made of silicon carbide, and a hole through which light passes is formed in the covering layer. However, in the configuration described in Japanese Patent No. 5736795, the temperature of a portion of the cell corresponding to the hole of the covering layer may become relatively low, and an alkali metal may be precipitated on an inner surface of the portion.

### SUMMARY

An object of one aspect of the present disclosure is to provide a cell unit capable of appropriately suppressing the precipitation of an alkali metal on an inner surface of a wall portion of a body portion of a cell through which light passes, when a heater is in operation.

A cell unit according to one aspect of the present disclosure is [1] “a cell unit including: a cell which includes a body portion through which light passes, and in which an alkali metal is sealed; a first heat conductive member that is at least partially disposed on the body portion; and a heater thermally connected to the first heat conductive member. The first heat conductive member covers an outer surface of a wall portion of the body portion through which the light passes, and is transmissive to the light”.

In the cell unit, the outer surface of the wall portion of the body portion of the cell through which the light passes is covered with the first heat conductive member that is transmissive to the light. Accordingly, since heat generated in the heater is transferred to the wall portion via the first heat conductive member, when the heater is in operation, a relative decrease in the temperature of the wall portion of the body portion of the cell through which the light passes is suppressed. Therefore, according to the cell unit, when the heater is in operation, the precipitation of the alkali metal on an inner surface of the wall portion of the body portion of the cell through which the light passes can be appropriately suppressed.

A cell unit according to one aspect of the present disclosure may be [2] “the cell unit according to [1] above in which the first heat conductive member is made of sapphire”. According to the cell unit, the first heat conductive member in which both heat transfer and light transmittance can be ensured.

A cell unit according to one aspect of the present disclosure may be [3] “the cell unit according to [1] or [2] above in which a thickness of the first heat conductive member in a direction in which the light passes is 0.1 mm or more and 1 mm or less”. According to the cell unit, sufficient heat transfer can be ensured by setting the thickness of the first heat conductive member to 0.1 mm or more, and an increase in the size of the cell unit can be suppressed by setting the thickness of the first heat conductive member to 1 mm or less.

A cell unit according to one aspect of the present disclosure may be [4] “the cell unit according to any one of [1] to [3] above in which the body portion and the first heat conductive member are bonded to each other via a first adhesive layer, and the first adhesive layer is transmissive to the light”. In this case, heat transfer and light transmittance between the body portion and the first heat conductive member can be ensured.

A cell unit according to one aspect of the present disclosure may be [5] “the cell unit according to any one of [1] to [4] above further includes a second heat conductive member that is at least partially disposed on the body portion. The heater is disposed on the second heat conductive member”. According to the cell unit, heat generated in the heater can be efficiently transferred to the body portion of the cell via the second heat conductive member.

A cell unit according to one aspect of the present disclosure may be [6] “the cell unit according to [5] above in which the second heat conductive member is made of sapphire or alumina”. According to the cell unit, the second heat conductive member in which at least heat transfer is ensured can be obtained.

A cell unit according to one aspect of the present disclosure may be [7] “the cell unit according to [5] or [6] above in which the first heat conductive member and the second heat conductive member are in contact with each other or are bonded to each other via a second adhesive layer”. According to the cell unit, since heat is directly transferred between the first heat conductive member and the second heat conductive member, heat generated in the heater can be efficiently transferred to the body portion of the cell.

According to one aspect of the present disclosure, it is possible to provide the cell unit capable of appropriately suppressing the precipitation of the alkali metal on the inner surface of the wall portion of the body portion of the cell through which the light passes, when the heater is in operation.

### BRIEF DESCRIPTION OF THE DRAWINGS

FIG. 1 is a view for describing the operation of an optically excited magnetic sensor module.

FIG. 2A is a perspective view of the optically excited magnetic sensor module, and FIG. 2B is a perspective view of the optically excited magnetic sensor module with an outer cover detached.

FIG. 3 is a perspective view of the optically excited magnetic sensor module in a state in which a housing lid portion is detached from a housing in FIG. 2B.

FIG. 4 is a cross-sectional view taken along line IV-IV in FIG. 3.

FIG. 5 is a plan view of the optically excited magnetic sensor module in a state in which a casing lid portion is detached from a cell casing in FIG. 3.

FIG. 6 is a perspective view of a portion of a cell unit.

FIG. 7 is a cross-sectional view taken along line VII-VII in FIG. 6.

FIG. 8 is a cross-sectional view taken along line VIII-VIII in FIG. 6.

FIG. 9 is a schematic plan view of a heater shown in FIG. 6.

FIGS. 10A and 10B are cross-sectional views of a cell unit according to a modification example.

FIG. 11 is a view for describing the operation of an optically excited magnetic sensor module according to a modification example.

### DETAILED DESCRIPTION

Hereinafter, one example of the present disclosure will be described in detail with reference to the drawings. Incidentally, in the drawings, the same or corresponding portions are denoted by the same reference signs, and duplicate descriptions will be omitted.

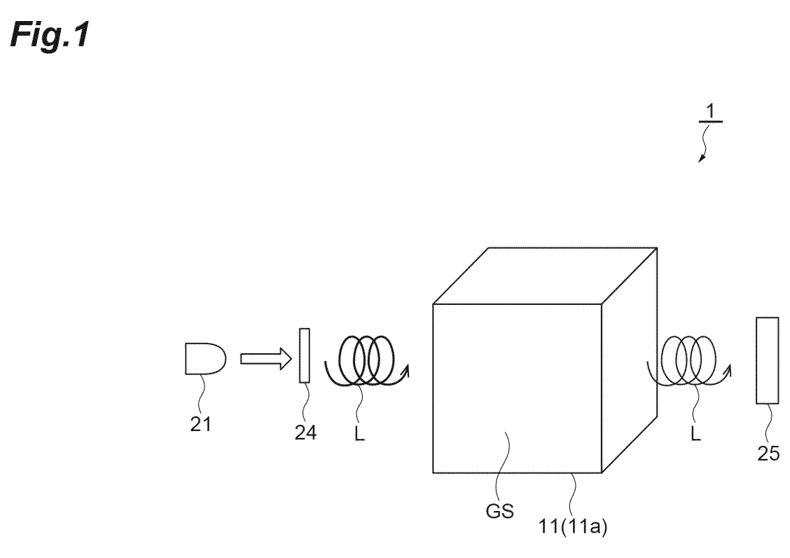

An optically excited magnetic sensor module 1 (hereinafter, also referred to as the “sensor module 1”) shown in FIGS. 1 to 5 is an optically pumped magnetometer (OPM), and is used, for example, for biomagnetic field measurements. As one example, the sensor module 1 can be used as a magnetoencephalograph that measures a magnetic field generated in the brain, or a magnetocardiograph or magnetospinograph that measures a magnetic field generated in the heart or spinal cord.

### Configuration of sensor module

As shown in FIGS. 1 to 5, the sensor module 1 includes a cell unit 2; a housing 3 that houses the cell unit 2; and an outer cover 4 that covers an outer surface of the housing 3. The cell unit 2 includes a cell 11, a plurality of heat conductive members 5 to 9, a heater 12, a heat insulating member 13, and a cell casing 14, and is at least partially surrounded by a coil unit 15. The sensor module 1 further includes a light source 21, a lens 22, a mirror 23, a quarter-wave plate 24, a photodetector 25, a connector member 26, and a connector cover 27.

### Operation of sensor module

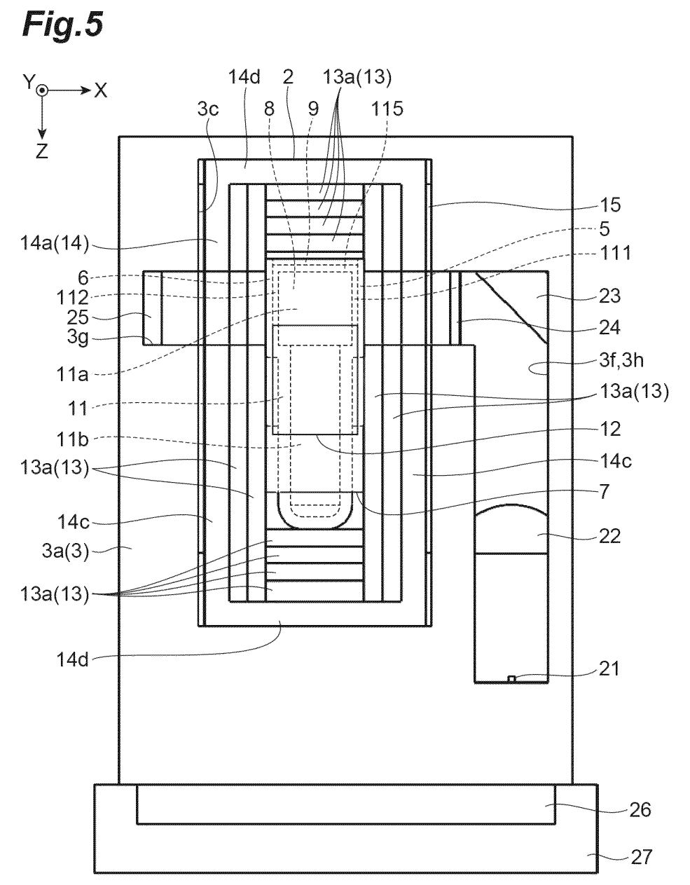

The operation of the sensor module 1 (the principle of detecting a change in magnetic field) will be described with reference to FIG. 1. During measurement, the sensor module 1 (cell 11) is disposed close to a measurement target. A gas GS containing an alkali metal is sealed in the cell 11. During measurement, the alkali metal inside the cell 11 is heated by the heater 12 (see FIG. 5), and the inside of the cell 11 is filled with alkali metal vapor. In this state, laser light L output from the light source 21 passes through the cell 11. The laser light L is incident on the cell 11 in a state in which the laser light L is converted into circularly polarized light by the quarter-wave plate 24. The circularly polarized laser light L brings the alkali metal vapor inside the cell 11 into a spin-polarized state through optical pumping (optical excitation). Namely, the laser light L functions as pump light that brings the alkali metal vapor inside the cell 11 into a spin-polarized state through optical pumping.

The laser light L that has passed through the cell 11 is detected by the photodetector 25. At this time, the intensity of the laser light L detected by the photodetector 25 (namely, the degree to which the laser light L is absorbed by the alkali metal vapor inside the cell 11) changes according to the spin-polarized state of the alkali metal vapor inside the cell 11. Here, the spin-polarized state of the alkali metal vapor inside the cell 11 changes under the influence of the magnetic field of the measurement target. Therefore, a change in the magnetic field of the measurement target can be detected based on the intensity of the detected laser light L. In this manner, the laser light L also functions as probe light for detecting the spin-polarized state of the alkali metal vapor inside the cell 11. In this case, the sensor module 1 is a single-laser sensor module in which the laser light L serves as both pump light and probe light.

### Configuration of each part of sensor module

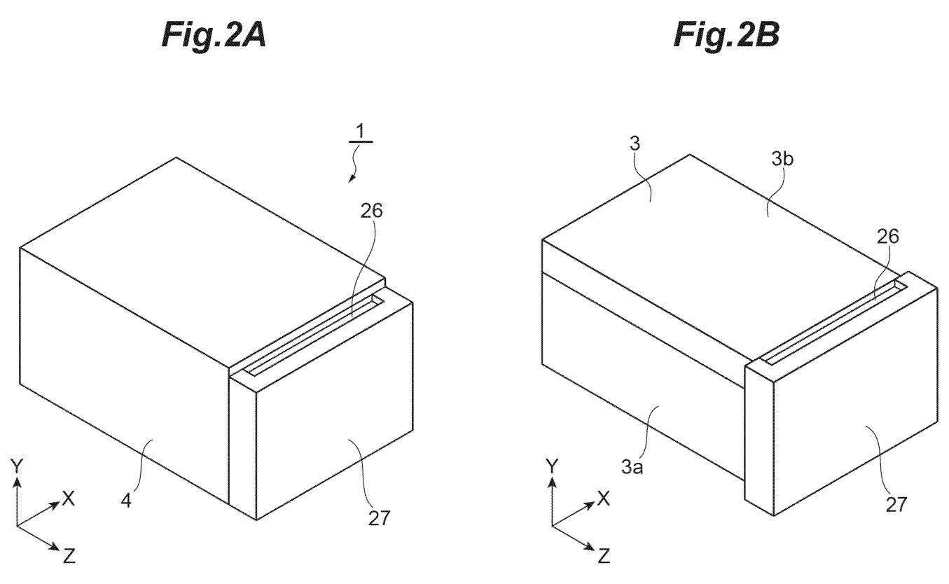

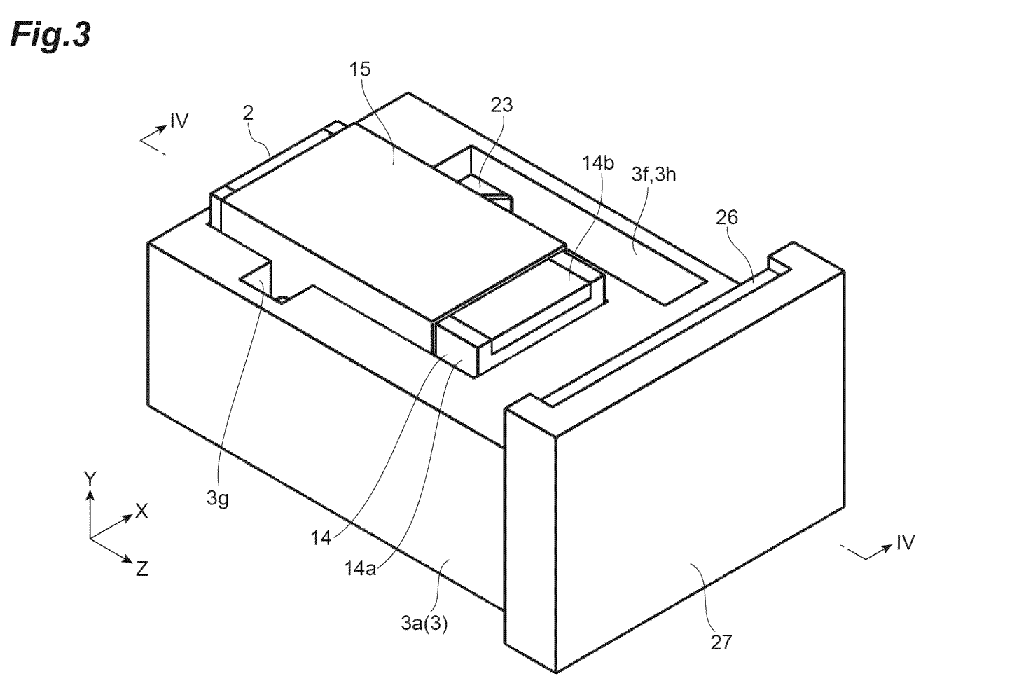

A configuration of each part of the sensor module 1 will be described with reference to FIGS. 2A to 5. Hereinafter, the description will be given with reference to an X direction, a Y direction perpendicular to the X direction, and a Z direction perpendicular to both the X direction and the Y direction shown in FIGS. 2A to 5. The housing 3 is formed, for example, from a resin material into a substantially rectangular parallelepiped shape, and includes a housing body portion 3a and a housing lid portion 3b. FIG. 2B shows a state in which the housing lid portion 3b is attached, and FIG. 3 shows a state in which the housing lid portion 3b is detached.

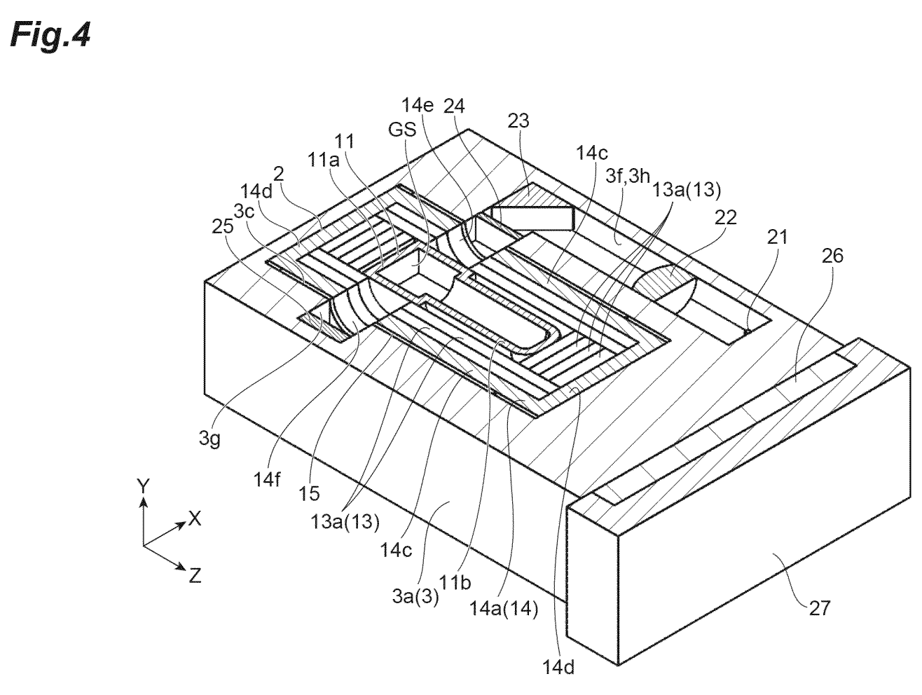

As shown in FIGS. 4 and 5, inside the housing 3, a cell unit disposition portion 3c in which the cell unit 2 is disposed, an optical member disposition portion 3f in which the light source 21, the lens 22, and the mirror 23 are disposed, and a photodetector disposition portion 3g in which the photodetector 25 is disposed are formed. An optical path portion 3h that is a space through which the laser light L output from the light source 21 travels toward the cell unit 2 is formed in the optical member disposition portion 3f. The photodetector disposition portion 3g is disposed on the side opposite the optical path portion 3h with respect to the cell unit disposition portion 3c.

The light source 21 is, for example, a vertical cavity surface emitting laser, and outputs the laser light L. In this example, the light source 21 is mounted on the connector member 26.

The lens 22 converts the laser light L output from the light source 21 into collimated light. The mirror 23 reflects the laser light L, which is collimated by the lens 22, toward the cell unit 2. The quarter-wave plate 24 is disposed between the mirror 23 and the cell unit 2. The quarter-wave plate 24 imparts a phase difference of π/2 (= λ/4) between the vertically polarized component of the incident light. The linearly polarized laser light L output from the light source 21 is converted into circularly polarized light by the quarter-wave plate 24.

The cell unit 2 is disposed in the cell unit disposition portion 3c. The cell unit 2 includes the cell 11 in which the gas GS containing an alkali metal is sealed. Details of the cell unit 2 will be described later. The laser light L that is converted into circularly polarized light by the quarter-wave plate 24 is incident on the cell unit 2. The laser light L incident on the cell unit 2 passes through the cell 11, and is emitted from the cell unit 2 toward the photodetector 25. In this example, the laser light L passes through the cell 11 along the X direction. The photodetector 25 is disposed in the photodetector disposition portion 3g. The photodetector 25 is, for example, a photodiode, and detects the laser light L that has passed through the cell unit 2.

The connector member 26 is provided on one side in the Z direction with respect to the housing 3. The heater 12, the coil unit 15, the light source 21, and the photodetector 25 are electrically connected to the connector member 26, and the connector member 26 is used to electrically connect these parts to the outside. The connector cover 27 is detachably attached to the connector member 26. As shown in FIGS. 2A and 2B, the outer cover 4 is formed in a substantially rectangular parallelepiped shape, and covers the outer surface of the housing 3 except for the surface on the side on which the connector cover 27 is disposed (the one side in the Z direction).

### Cell unit

Details of the cell unit 2 will be described with reference to FIGS. 3 to 5. As described above, the cell unit 2 includes the cell 11, the plurality of heat conductive members 5 to 9, the heater 12, the heat insulating member 13, and the cell casing 14, and is at least partially surrounded by the coil unit 15. The cell 11, the plurality of heat conductive members 5 to 9, the heater 12, and the heat insulating member 13 are housed in the cell casing 14, and the coil unit 15 is disposed outside the cell casing 14.

The cell 11 includes a body portion 11a and a protruding portion 11b. The cell 11 is made of, for example, a light-transmitting material such as glass or silicon. The gas GS consisting of an alkali metal and an inert gas is sealed in the cell 11. The alkali metal sealed in the cell 11 is, for example, one or a plurality of potassium, lithium, sodium, rubidium, and cesium. The inert gas sealed in the cell 11 is, for example, one or a plurality of helium, neon, argon, krypton, xenon, nitrogen, and hydrogen. The body portion 11a is, for example, a rectangular parallelepiped container portion. The protruding portion 11b is a tubular portion connected to the body portion 11a, and is a portion that is sealed off after being mainly used as a passage or the like for gas introduction and discharge such as gas exhaust and the introduction of the gas GS.

The plurality of heat conductive members 5 to 9 cover the body portion 11a of the cell 11. The heater 12 is disposed on the heat conductive member 7. The heater 12 is thermally connected to the cell 11 via the heat conductive member 7. The heater 12 includes, for example, a heating wire (a resistor such as a metal wire) that generates heat when energized, and is formed in a sheet shape. The heater 12 generates heat when energized, thereby heating the cell 11 via the plurality of heat conductive members 5 to 9. Details of the plurality of heat conductive members 5 to 9 and the heater 12 will be described later.

The heat insulating member 13 is disposed inside the cell casing 14 to be located outside the cell 11, the plurality of heat conductive members 5 to 9, and the heater 12 (hereinafter, referred to as the “cell 11 and the like”). In this example, the heat insulating member 13 is composed of a plurality of plate-shaped members 13a. The plurality of plate-shaped members 13a are disposed to fill spaces between the cell 11 and the like and the cell casing 14. In more detail, the plurality of plate-shaped members 13a are disposed in a space between the plurality of heat conductive members 5, 6, and 9 that cover the body portion 11a of the cell 11 and the cell casing 14 to fill the space with as little gaps as possible, except for a region of the body portion 11a through which the laser light L transmits, and are disposed in a space between the protruding portion 11b of the cell 11 and the cell casing 14 to surround the entirety of the protruding portion 11b and fill the space while having a region spaced apart from an outer surface of the protruding portion 11b of the cell 11. Each of the plate-shaped members 13a is, for example, a member, the heat insulation of which is enhanced by forming an air layer inside. The cell casing 14 is formed, for example, from a resin material into a substantially rectangular parallelepiped shape, and includes a casing body portion 14a and a casing lid portion 14b. FIG. 3 shows a state in which the casing lid portion 14b is attached, and FIG. 5 shows a state in which the casing lid portion 14b is detached. Incidentally, the hatching of the plate-shaped members 13a is omitted in FIG. 4.

As shown in FIG. 5, the casing body portion 14a includes a pair of first wall portions 14c facing each other in the X direction, and a pair of second wall portions 14d facing each other in the Z direction. A plurality of (in this example, two) plate-shaped members 13a are disposed between each of the first wall portions 14c and the cell 11 and the like, and a plurality of (in this example, four) plate-shaped members 13a are disposed between each of the second wall portions 14d and the cell 11 and the like. Although not shown, the heat insulating member 13 (a plurality of the plate-shaped members 13a) is also disposed between a wall portion of the casing body portion 14a in the Y direction (a wall portion facing the casing lid portion 14b in the Y direction) and the cell 11 and the like, and between the casing lid portion 14b and the cell 11 and the like. In this manner, in this example, the cell 11 and the like are housed in the cell casing 14 in a state in which the heat insulating member 13 is interposed between the cell casing 14 and the cell 11 and the like. As shown in FIG. 4, an opening 14e through which the laser light L reflected by the mirror 23 and traveling toward the cell 11 passes is formed in one of the pair of first wall portions 14c, and an opening 14f through which the laser light L that has passed through the cell 11 passes is formed in the other of the pair of first wall portions 14c.

As shown in FIG. 3, the coil unit 15 is disposed outside the cell casing 14. The coil unit 15 includes, for example, a plurality of coils, and a magnetic field acting on the cell 11 is generated by the coils. In the sensor module 1, while a magnetic field acting on the cell 11 is generated by the coil unit 15, a change in the magnetic field inside the cell 11 is detected using the laser light L (probe light).

The coil unit 15 generates, for example, a correction magnetic field such that the influence of magnetic fields other than the magnetic field of the measurement target on the cell 11 approaches zero. For example, the coil unit 15 may generate a magnetic field in a direction opposite a geomagnetic field such that the influence of the geomagnetic field is cancelled out. Instead thereof or in addition thereto, the coil unit 15 may generate a modulated magnetic field acting on the cell 11. For example, the coil unit 15 may generate an alternating magnetic field modulated at a predetermined frequency for increased sensitivity, or may generate a modulated magnetic field for enabling detection of the direction of a magnetic field change (positive and negative directions on each axis). In this example, the coil unit 15 is composed of a flexible circuit board, is disposed to surround four sides of the cell casing 14 (cell 11) (sides of the cell casing 14 other than the second wall portions 14d) (sides of the cell 11 other than two sides in an extending direction of the protruding portion 11b), and is led out to the one side in the Z direction and electrically connected to the connector member 26.

### Plurality of heat conductive members and heater

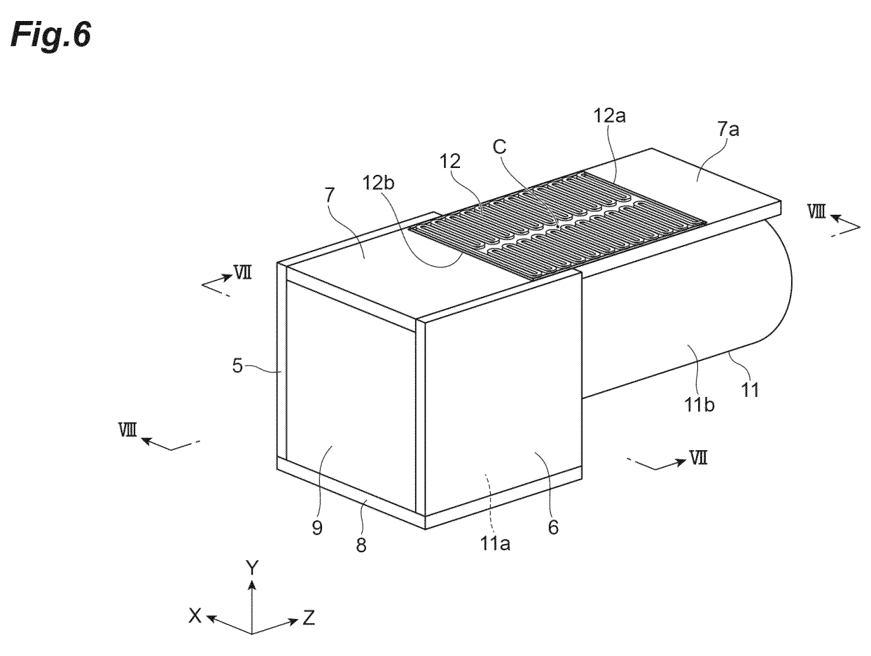

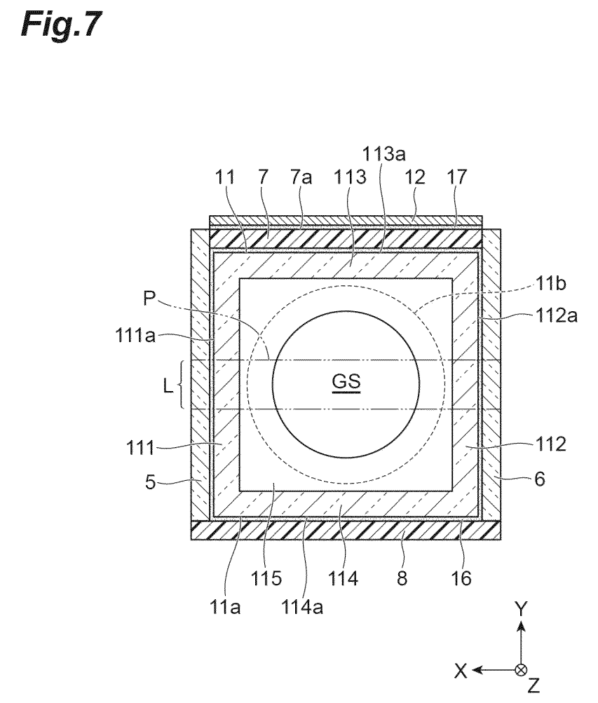

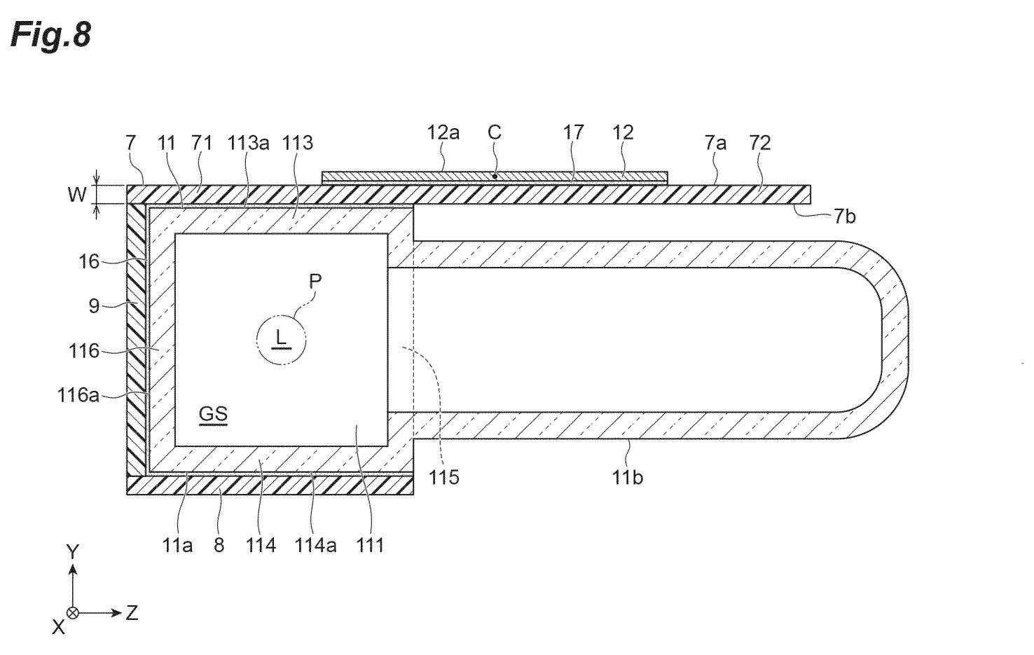

As shown in FIGS. 6 to 8, in the cell 11, the body portion 11a includes a wall portion 111 on one side in the X direction; a wall portion 112 on the other side in the X direction; a wall portion 113 on one side in the Y direction; a wall portion 114 on the other side in the Y direction; a wall portion 115 on the one side in the Z direction; and a wall portion 116 on the other side in the Z direction. The protruding portion 11b extends from the wall portion 115 to the one side in the Z direction. In the cell 11, the laser light (light) L is incident on the body portion 11a through the wall portion 111, passes through the gas GS inside the body portion 11a, and is emitted to the outside of the body portion 11a through the wall portion 112. In this manner, the laser light L passes through the body portion 11a. An optical path P of the laser light L extends in the X direction in the body portion 11a. The width (beam diameter) of the optical path P of the laser light L is determined by, for example, the lens 22 (see FIG. 5).

The heat conductive member (first heat conductive member) 5 is bonded to an outer surface 111a of the wall portion 111 via an adhesive layer 16 (first adhesive layer). The heat conductive member (first heat conductive member) 6 is bonded to an outer surface 112a of the wall portion 112 via the adhesive layer 16. The heat conductive member (second heat conductive member) 7 is bonded to an outer surface 113a of the wall portion 113 via the adhesive layer 16. The heat conductive member 8 is bonded to an outer surface 114a of the wall portion 114 via the adhesive layer 16. The heat conductive member 9 is bonded to an outer surface 116a of the wall portion 116 via the adhesive layer 16. Incidentally, the adhesive layer 16 is transmissive to the laser light L. However, only the adhesive layers 16 for the heat conductive member 5 and the heat conductive member 6 through which the laser light L passes must be transmissive to the laser light L, and the adhesive layers 16 for the heat conductive member 7, the heat conductive member 8, and the heat conductive member 9 through which the laser light L does not pass may not be transmissive to the laser light L. In this manner, each of the heat conductive members 5 to 9 is disposed on the body portion 11a. Incidentally, each of the heat conductive members 5 to 9 may be disposed on the body portion 11a to abut against the body portion 11a without the adhesive layer 16 interposed therebetween.

Each of the heat conductive members 5 to 9 is formed from a non-conductive material into a plate shape (for example, a rectangular plate shape). The non-conductive material is a material having low electrical conductivity, namely, a so-called electrically insulating material. The heat conductive member 5 that covers the outer surface 111a of the wall portion 111 that is a wall portion of the body portion 11a through which the laser light L passes is transmissive to the laser light L. Similarly, the heat conductive member 6 that covers the outer surface 112a of the wall portion 112 that is a wall portion of the body portion 11a through which the laser light L passes is transmissive to the laser light L. In addition, the heat conductive members 7, 8, and 9 that cover the outer surfaces 113a, 114a, and 116a of the wall portions 113, 114, and 116 that are not the wall portions of the body portion 11a through which the laser light L passes may also be transmissive to the laser light L, but may not be transmissive to the laser light L. In this example, each of the heat conductive members 5 and 6 is made of sapphire, and each of the heat conductive members 7 to 9 is made of sapphire or alumina. A thickness of each of the heat conductive members 5 to 9 is 0.1 mm or more and 1 mm or less. A thermal conductivity of the material of each of the heat conductive members 5 to 9 is higher than a thermal conductivity of at least a material constituting each of the wall portions 111 and 112 of the body portion 11a through which the laser light L passes. Namely, in the present embodiment, the heat conductive member is a member having a thermal conductivity higher than the thermal conductivity of at least the material constituting each of the wall portions 111 and 112 of the body portion 11a through which the laser light L passes.

The heat conductive member 5 is in contact with the heat conductive members 7 and 8 at both edges in the Y direction, and is in contact with the heat conductive member 9 at an edge on the other side in the Z direction. The heat conductive member 6 is in contact with the heat conductive members 7 and 8 at both edges in the Y direction, and is in contact with the heat conductive member 9 at an edge on the other side in the Z direction. The heat conductive member 7 is in contact with the heat conductive members 5 and 6 at both edges in the X direction, and is in contact with the heat conductive member 9 at an edge on the other side in the Z direction. The heat conductive member 8 is in contact with the heat conductive members 5 and 6 at both edges in the X direction, and is in contact with the heat conductive member 9 at an edge on the other side in the Z direction. The heat conductive member 9 is in contact with the heat conductive members 5 and 6 at both edges in the X direction, and is in contact with the heat conductive members 7 and 8 at both edges in the Y direction. In this manner, the heat conductive members adjacent to each other among the plurality of heat conductive members 5 to 9 are in contact with each other. Namely, the entirety of the body portion 11a is surrounded by the heat conductive members 5 to 9, except for the wall portion 115 on which the protruding portion 11b is provided. The heat conductive members adjacent to each other among the plurality of heat conductive members 5 to 9 may be bonded to each other via a second adhesive layer. In the present embodiment, the second adhesive layer includes a part of the adhesive layer 16. Incidentally, the second adhesive layer may not include the adhesive layer 16, and may be made of a material different from that of the adhesive layer 16. In this case, the second adhesive layer may not be transmissive to the laser light L.

The heater 12 is bonded to a surface 7a of the heat conductive member 7, which is on the side opposite the cell 11, via an adhesive layer 17. In this manner, the heater 12 is disposed on the heat conductive member 7. The heater 12 is thermally connected to the plurality of heat conductive members 5 to 9. The heater 12 has a heating region 12a that generates heat when energized. The heating region 12a is a layer-shaped region, the thickness direction of which is aligned with the Y direction, and the shape of the heating region 12a when viewed in the Y direction is, for example, a rectangular shape. Details of the heating region 12a will be described later.

When viewed in the Y direction that is a direction in which the body portion 11a and the heat conductive member 7 overlap each other, a center C of the heating region 12a overlaps the heat conductive member 7, and is shifted from the optical path P of the laser light L in the Z direction that is a direction intersecting the Y direction. Namely, when viewed in the Y direction, the center C of the heating region 12a is spaced apart from the optical path P of the laser light L in the Z direction. In this example, when viewed in the Y direction, the center C of the heating region 12a is shifted to the one side in the Z direction from the optical path P of the laser light L by an amount equal to or larger than a width (thickness) W of the heat conductive member 7 in the Y direction. When viewed in the Y direction, the heating region 12a is disposed on the heat conductive member 7 across a first portion 71 and a second portion 72 of the heat conductive member 7, and the center C of the heating region 12a overlaps the second portion 72 of the heat conductive member 7. Namely, the center C of the heating region 12a is disposed on the second portion 72 of the heat conductive member 7. The first portion 71 is a portion of the heat conductive member 7 that is located on the body portion 11a. The second portion 72 is a portion of the heat conductive member 7 that extends from the first portion 71 to the one side in the Z direction. In the second portion 72, a gap is formed between a surface 7b on the cell 11 side of the heat conductive member 7 and an outer surface of the protruding portion 11b. Incidentally, the center C of the heating region 12a refers to the center of gravity of the shape of the heating region 12a when viewed in the Y direction. In addition, in the present embodiment, the entirety of the heating region 12a is shifted to the one side in the Z direction from the optical path P of the laser light L (there is no overlapping region when viewed in the Y direction); however, the heating region 12a may include a region overlapping the optical path P when viewed in the Y direction to the extent that the influence of noise can be suppressed. For example, an end portion 12b of the heating region 12a on the other side in the Z direction may overlap the optical path P when viewed in the Y direction.

In addition, when viewed in the Y direction, the entirety of the heating region 12a is shifted to the one side in the Z direction from the optical path P of the laser light L, and at least a part of the heating region 12a is located inside the body portion 11a. Specifically, when viewed in the Y direction, the end portion 12b of the heating region 12a on the other side in the Z direction is shifted to the one side in the Z direction from the optical path P of the laser light L, and is located inside the body portion 11a. In addition, in the present embodiment, the entirety of the heating region 12a is disposed on the heat conductive member 7.

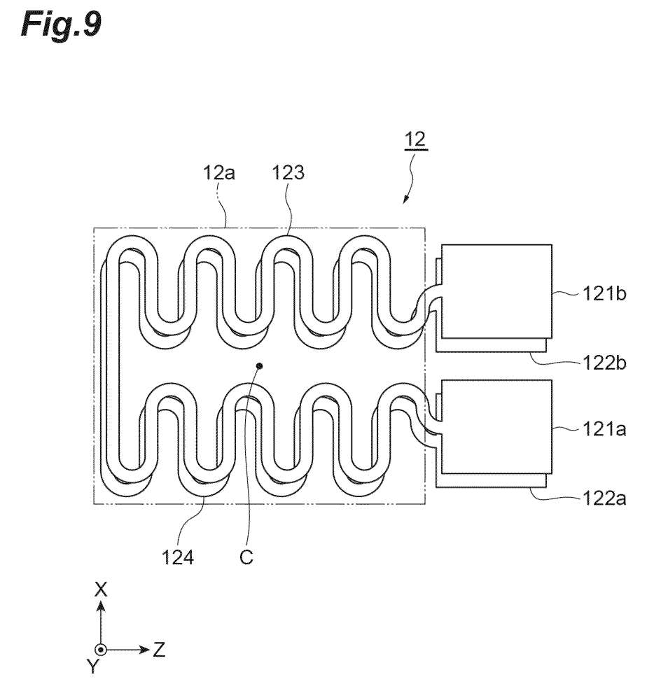

As shown in FIG. 9, the heater 12 includes a pair of first electrode portions 121a and 121b, a pair of second electrode portions 122a and 122b, a first heating wire 123, and a second heating wire 124. One end of the first heating wire 123 is connected to the first electrode portion 121a, and the other end of the first heating wire 123 is connected to the first electrode portion 121b. One end of the second heating wire 124 is connected to the second electrode portion 122a, and the other end of the second heating wire 124 is connected to the second electrode portion 122b. Each of the first heating wire 123 and the second heating wire 124 is a heating wire (a resistor such as a metal wire) that generates heat when energized.

In this example, the pair of first electrode portions 121a and 121b are arranged side by side in the X direction. The first heating wire 123 extends in a meandering shape or a zigzag shape from the first electrode portion 121a to the other side in the Z direction, and extends in a meandering shape or a zigzag shape from the other side in the Z direction to the first electrode portion 121b. Similarly, the pair of second electrode portions 122a and 122b are arranged side by side in the X direction. The second heating wire 124 extends in a meandering shape or a zigzag shape from the second electrode portion 122a to the other side in the Z direction, and extends in a meandering shape or a zigzag shape from the other side in the Z direction to the second electrode portion 122b.

The first heating wire 123 and the second heating wire 124 extend while being arranged side by side. Namely, the first heating wire 123 and the second heating wire 124 are spaced apart from each other in a predetermined direction, and overlap each other when viewed in the predetermined direction. In this example, the first heating wire 123 and the second heating wire 124 are spaced apart from each other in the Y direction, and overlap each other when viewed in the Y direction. The first heating wire 123 and the second heating wire 124 is electrically insulated from each other, for example, by being covered with a sheet (not shown) having heat resistance and electrical insulation. In the heater 12, the heating region 12a is defined by the first heating wire 123 and the second heating wire 124. In this example, the heating region 12a is a rectangular parallelepiped region of the minimum volume that includes the first heating wire 123 and the second heating wire 124 extending in a meandering shape or a zigzag shape.

When the heater 12 is in operation, an electric current flows through the first heating wire 123 from the first electrode portion 121a toward the first electrode portion 121b, and an electric current flows through the second heating wire 124 from the second electrode portion 122b toward the second electrode portion 122a. Accordingly, in portions overlapping each other when viewed in the Y direction (namely, portions arranged side by side), the direction of the electric current flowing through the first heating wire 123 and the direction of the electric current flowing through the second heating wire 124 are opposite each other. In this manner, the first heating wire 123 and the second heating wire 124 generate heat when energized in the opposite directions. In this case, a magnetic field generated in the first heating wire 123 due to energization and a magnetic field generated in the second heating wire 124 due to energization cancel each other out. Incidentally, for that purpose, a distance between the portions of the first heating wire 123 and the second heating wire 124 that are arranged side by side is 1 mm or less.

### Actions and effects

In the cell unit 2, the outer surface 111a of the wall portion 111 of the body portion 11a of the cell 11 through which the laser light L passes is covered with the heat conductive member 5 that is transmissive to the laser light L. Similarly, the outer surface 112a of the wall portion 112 of the body portion 11a of the cell 11 through which the laser light L passes is covered with the heat conductive member 6 that is transmissive to the laser light L. Accordingly, since heat generated in the heater 12 is transferred to the wall portions 111 and 112 via the respective heat conductive members 5 and 6, when the heater 12 is in operation, a relative decrease in the temperature of each of the wall portions 111 and 112 of the body portion 11a of the cell 11 through which the laser light L passes is suppressed. Therefore, according to the cell unit 2, when the heater 12 is in operation, the precipitation of the alkali metal on an inner surface of each of the wall portions 111 and 112 of the body portion 11a of the cell 11 through which the laser light L passes can be appropriately suppressed. In addition, since each of the heat conductive members 5 and 6 is made of a non-conductive material, the generation of magnetic noise in each of the heat conductive members 5 and 6 that are heated can be suppressed compared to, for example, when each of the heat conductive members 5 and 6 is made of a conductive material.

In the cell unit 2, each of the heat conductive members 5 and 6 is made of sapphire. Accordingly, each of the heat conductive members 5 and 6 in which both heat transfer (thermal conductivity) and light transmittance are ensured.

In the cell unit 2, the thickness of each of the heat conductive members 5 and 6 in the X direction through which the laser light L passes is 0.1 mm or more and 1 mm or less. Sufficient heat transfer can be ensured by setting the thickness of each of the heat conductive members 5 and 6 to 0.1 mm or more, and an increase in the size of the cell unit 2 can be suppressed by setting the thickness of each of the heat conductive members 5 and 6 to 1 mm or less.

In the cell unit 2, the adhesive layer 16 is transmissive to light. Accordingly, heat transfer and light transmittance between the body portion 11a of the cell 11 and the heat conductive member 5 and between the body portion 11a and the heat conductive member 6 can be ensured.

In the cell unit 2, the heat conductive member 7 made of a non-conductive material is disposed on the body portion 11a of the cell 11, and the heater 12 is disposed on the heat conductive member 7. Accordingly, heat generated in the heater 12 can be efficiently transferred to the body portion 11a of the cell 11 via the heat conductive member 7. In addition, since the heat conductive member 7 is made of a non-conductive material, the generation of magnetic noise in the heat conductive member 7 that is heated can be suppressed compared to, for example, when the heat conductive member 7 is made of a conductive material.

In the cell unit 2, the heat conductive member 7 is made of sapphire or alumina. Accordingly, the heat conductive member 7 in which at least heat transfer is ensured.

In the cell unit 2, the heat conductive member 5 and the heat conductive member 7 are in contact with each other, and the heat conductive member 6 and the heat conductive member 7 are in contact with each other. Alternatively, the heat conductive member 5 and the heat conductive member 7 are bonded to each other via the adhesive layer 16, and the heat conductive member 6 and the heat conductive member 7 are bonded to each other via the adhesive layer 16. In both cases, since heat is directly transferred between each of the heat conductive members 5 and 6 and the heat conductive member 7, heat generated in the heater 12 can be efficiently transferred to the body portion 11a of the cell 11.

In the cell unit 2, the heat conductive member 7 is disposed on the body portion 11a of the cell 11, and the heater 12 is disposed on the heat conductive member 7 in a state in which the center C of the heating region 12a of the heater 12 overlaps the heat conductive member 7 when viewed in the Y direction. Accordingly, heat generated in the heater 12 can be efficiently transferred to the body portion 11a of the cell 11 via the heat conductive member 7. In addition, since the heat conductive member 7 is made of a non-conductive material, the generation of magnetic noise in the heat conductive member 7 that is heated can be suppressed compared to, for example, when the heat conductive member 7 is made of a conductive material. Furthermore, since the center C of the heating region 12a of the heater 12 is shifted from the optical path P of the laser light L in the Z direction when viewed in the Y direction, the influence of magnetic noise generated in the heating region 12a due to energization can be suppressed. Therefore, according to the cell unit 2, heat generated in the heater 12 can be efficiently transferred to the body portion 11a of the cell 11 while suppressing the influence of magnetic noise on measurements in the body portion 11a of the cell 11.

In the cell unit 2, the body portion 11a of the cell 11 is covered with the plurality of heat conductive members 5 to 9. Accordingly, heat generated in the heater 12 can be efficiently transferred to the body portion 11a of the cell 11 via the plurality of heat conductive members 5 to 9, and the body portion 11a can be uniformly heated. In addition, since each of the heat conductive members 5 to 9 is made of a non-conductive material, the generation of magnetic noise in each of the heat conductive members 5 to 9 that are heated can be suppressed.

In the cell unit 2, when viewed in the Y direction, the center C of the heating region 12a is shifted from the optical path P of the laser light L in the Z direction by an amount equal to or larger than the width W of the heat conductive member 7 in the Y direction. Accordingly, the influence of magnetic noise generated in the heating region 12a due to energization can be reliably suppressed.

In the cell unit 2, when viewed in the Y direction, the entirety of the heating region 12a is shifted from the optical path P of the laser light L in the Z direction. Accordingly, the influence of magnetic noise generated in the heating region 12a due to energization can be more reliably suppressed.

In the cell unit 2, when viewed in the Y direction, the entirety of the heating region 12a is shifted from the optical path P of the laser light L in the Z direction, and the end portion 12b of the heating region 12a is located inside the body portion 11a. Accordingly, the influence of magnetic noise on measurements in the body portion 11a can also be suppressed while more efficiently transferring heat generated in the heater 12 to the body portion 11a.

In the cell unit 2, in the cell 11, the protruding portion 11b extends from the body portion 11a to the one side in the Z direction, and the center C of the heating region 12a is shifted to the one side in the Z direction from the optical path P of the laser light L. Accordingly, an increase in the area of the cell unit 2 when viewed in the Y direction can be suppressed.

In the cell unit 2, the center C of the heating region 12a is disposed on the second portion 72. Accordingly, since the center C of the heating region 12a of the heater 12 can be set away from the optical path P of the laser light L while ensuring a heat transfer path, the influence of magnetic noise generated in the heating region 12a due to energization can be reliably suppressed.

In the cell unit 2, in the heater 12, the first heating wire 123 and the second heating wire 124 that define the heating region 12a extend while being arranged side by side, and the first heating wire 123 and the second heating wire 124 that define the heating region 12a generate heat when energized in the opposite directions. Accordingly, since a magnetic field generated in the first heating wire 123 due to energization and a magnetic field generated in the second heating wire 124 due to energization cancel each other out, the generation of magnetic noise in the heating region 12a due to energization can be suppressed.

Incidentally, in an example in which the body portion 11a of the cell 11 was not covered with any member, the sensitivity was 45.1 fT/rt Hz under predetermined conditions. On the other hand, in an example in which the body portion 11a of the cell 11 was covered with aluminum foil, the sensitivity was 147 fT/rt Hz under the predetermined conditions. From this result, it can be seen that, when the body portion 11a of the cell 11 is covered with a member made of a conductive material, magnetic noise increases with respect to signals and the sensitivity decreases. In addition, in an example in which the body portion 11a of the cell 11 was not covered with any member, a temperature difference occurring in the body portion 11a under predetermined conditions was 23.5°C. On the other hand, in an example in which the body portion 11a of the cell 11 was covered with the plurality of heat conductive members 5 to 9, a temperature difference occurring in the body portion 11a under predetermined conditions was 10.5°C. From this result, it can be seen that, when the body portion 11a of the cell 11 is covered with the plurality of heat conductive members 5 to 9, the body portion 11a is sufficiently uniformly heated.

### Modification examples

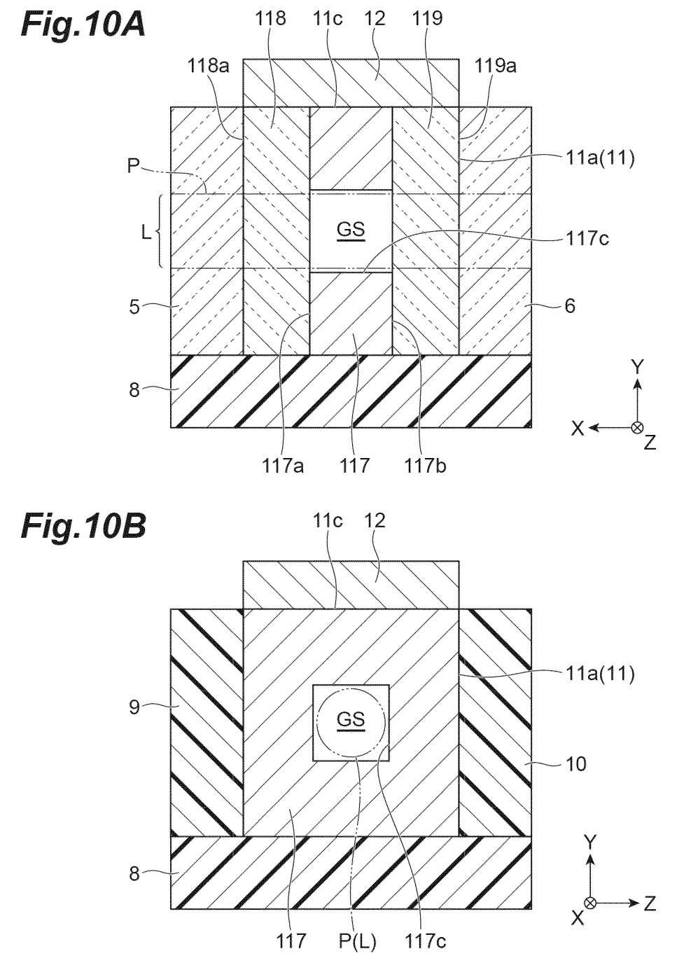

The present disclosure is not limited to one example described above. For example, as shown in FIGS. 10A and 10B, the body portion 11a of the cell 11 may be composed of an intermediate member 117 and a pair of light-transmitting members 118 and 119. FIG. 10A is a cross-sectional view perpendicular to the Z direction, and FIG. 10B is a cross-sectional view perpendicular to the X direction.

In the example shown in FIGS. 10A and 10B, the intermediate member 117 has a pair of surfaces 117a and 117b facing each other in the X direction, and a through-hole 117c that opens to each of the surfaces 117a and 117b. The intermediate member 117 is formed, for example, from silicon into a rectangular frame shape. The light-transmitting member 118 is disposed on the surface 117a of the intermediate member 117, and covers the through-hole 117c from the one side in the X direction. The light-transmitting member 119 is disposed on the surface 117b of the intermediate member 117, and covers the through-hole 117c from the other side in the X direction. Each of the light-transmitting members 118 and 119 is formed, for example, from glass into a rectangular plate shape. The intermediate member 117 and the light-transmitting member 118 are bonded together, for example, by anodic bonding or direct bonding. Similarly, the intermediate member 117 and the light-transmitting member 119 are bonded together, for example, by anodic bonding or direct bonding. The respective side surfaces of the intermediate member 117 and each of the pair of light-transmitting members 118 and 119 are flush with each other, and constitute a side surface 11c of the body portion 11a.

In the example shown in FIGS. 10A and 10B, the heat conductive member 5 is disposed on an outer surface 118a of the light-transmitting member 118 on the side opposite the intermediate member 117. The heat conductive member 6 is disposed on an outer surface 119a of the light-transmitting member 119 on the side opposite the intermediate member 117. The heater 12 is disposed on one of the side surfaces 11c of the body portion 11a on the one side in the Y direction. The heat conductive member 8 is disposed on one of the side surfaces 11c of the body portion 11a on the other side in the Y direction. The heat conductive member 10 is disposed on one of the side surfaces 11c of the body portion 11a on the one side in the Z direction. The heat conductive member 9 is disposed on one of the side surfaces 11c of the body portion 11a on the other side in the Z direction.

In the cell unit 2 including the body portion 11a of the cell 11 as described above as well, when the heater 12 is in operation, the precipitation of the alkali metal on an inner surface of each of the light-transmitting members 118 and 119 that are the wall portions of the body portion 11a of the cell 11 through which the laser light L passes can be appropriately suppressed.

The present invention is not limited to the example described above, and when viewed in the Y direction, the center C of the heating region 12a of the heater 12 may be shifted from the optical path P of the laser light L in a direction other than “the one side in the Z direction”, or may overlap the optical path P of the laser light L. Each of the heat conductive members 5 and 6 may be made of a “non-conductive material that is transmissive to the laser light L” other than sapphire. Each of the heat conductive members 7 to 9 may be made of a non-conductive material other than sapphire and alumina. Each of the heat conductive members 7 to 9 may not be made of a non-conductive material, but may be made of, for example, a semiconductor material or a conductive material. The entirety of each of the heat conductive members 5 to 9 may not be disposed on the body portion 11a of the cell 11, but it is sufficient if at least a part of each thereof is disposed on the body portion 11a of the cell 11. The thickness of each of the heat conductive members 5 to 9 may be smaller than 0.1 mm or may be larger than 1 mm.

In the example described above, the entirety of the heating region 12a of the heater 12 is disposed on the heat conductive member 7; however, it is sufficient if at least a part of the heating region 12a is disposed on the heat conductive member 7. In this case, the center C of the heating region 12a may not overlap the heat conductive member 7. The heat conductive members 5 and 6 may not be transmissive to the laser light L. In this case, the heat conductive members 5 and 6 may include window portions through which the laser light L passes.

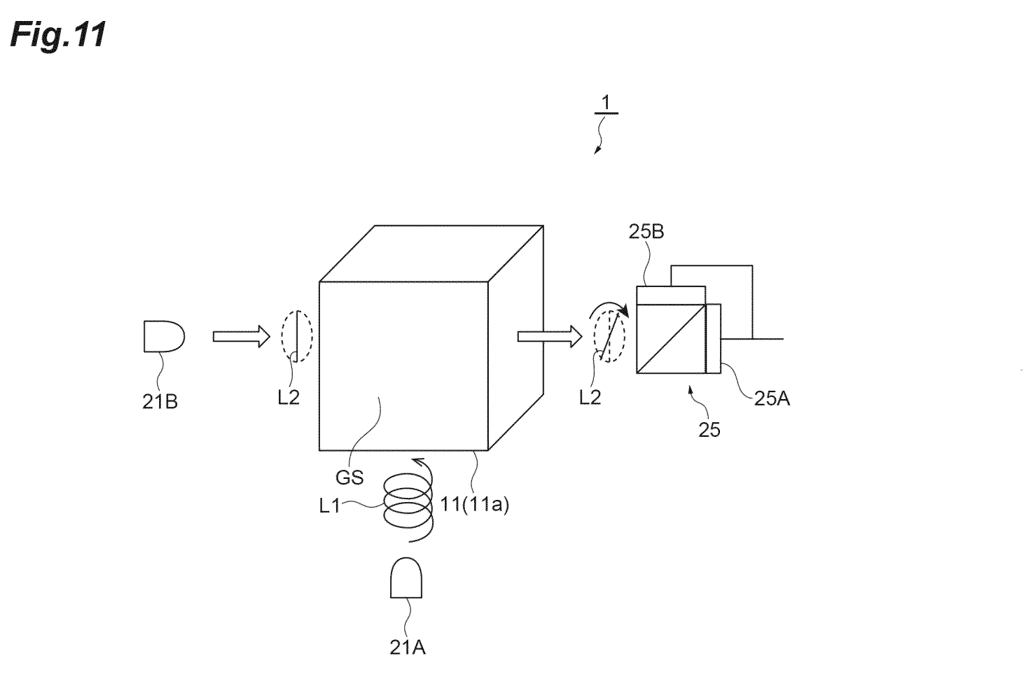

In the example described above, the sensor module 1 is configured as a single-laser sensor module in which the laser light L serves as both pump light and probe light; however, as in a modification example shown in FIG. 11, the sensor module 1 may be configured as a dual-laser sensor module in which pump light L1 and probe light L2 are independent. The sensor module 1 of the modification example includes a light source 21A that outputs the linearly polarized pump light L1, and a light source 21B that outputs the circularly polarized probe light L2, instead of the light source 21. In addition, in the sensor module 1 of the modification example, the photodetector 25 is configured as a differential detector composed of a first photodetector 25A and a second photodetector 25B.

In the sensor module 1 of the modification example, the pump light L1 output from the light source 21A brings the alkali metal vapor inside the cell 11 into a spin-polarized state through optical pumping. The probe light L2 output from the light source 21B and passing through the cell 11 is detected by the photodetector 25. Here, since the spin-polarized state of the alkali metal vapor inside the cell 11 is changed under the influence of the magnetic field of the measurement target, the polarization direction of the probe light L2 that has passed through the alkali metal vapor is changed to be tilted. Further, the first photodetector 25A detects the intensity of light in a deflection direction component corresponding to the polarization direction of the probe light L2 before the polarization direction is changed, and the second photodetector 25B detects the intensity of a deflection direction component corresponding to the polarization direction of the probe light L2 after the polarization direction is changed. Accordingly, a difference in light intensity between the two polarization direction components of the probe light L2 is detected. The spin-polarized state of the alkali metal vapor inside the cell 11 can be detected based on the difference, and therefore, a change in the magnetic field of the measurement target can be detected. In the case of a dual-laser sensor module, for example, the probe light L2 passes through the body portion 11a of the cell 11 along the X direction. The pump light L1 passes through the body portion 11a of the cell 11 along the Y direction.

The cell unit 2 is not limited to be applied to an optically pumped magnetometer such as the sensor module 1, and can also be applied to other devices such as an atomic clock.

What is claimed is:

1. A cell unit, comprising:
a cell which includes a body portion through which light passes, and in which an alkali metal is sealed;
a first heat conductive member that is at least partially disposed on the body portion; and
a heater thermally connected to the first heat conductive member,
wherein the first heat conductive member covers an outer surface of a wall portion of the body portion through which the light passes, and is transmissive to the light.

2. The cell unit according to claim 1,
wherein the first heat conductive member is made of sapphire.

3. The cell unit according to claim 1,
wherein a thickness of the first heat conductive member in a direction in which the light passes is 0.1 mm or more and 1 mm or less.

4. The cell unit according to claim 1,
wherein the body portion and the first heat conductive member are bonded to each other via a first adhesive layer, and
the first adhesive layer is transmissive to the light.

5. The cell unit according to claim 1, further comprising:
a second heat conductive member that is at least partially disposed on the body portion,
wherein the heater is disposed on the second heat conductive member.

6. The cell unit according to claim 5,
wherein the second heat conductive member is made of sapphire or alumina.

7. The cell unit according to claim 5,
wherein the first heat conductive member and the second heat conductive member are in contact with each other or are bonded to each other via a second adhesive layer.
